(19) United States
(12) Patent Application Publication
(10) Pub. No.: US 2015/0359912 A1
(43) Pub. Date: Dec. 17, 2015

(54) J-Aggregate Forming Nanoparticle

(71) Applicant: UNIVERSITY HEALTH NETWORK, Toronto (CA)

(72) Inventors: Gang Zheng, Toronto (CA); Elizabeth Huynh, Mississauga (CA); Kenneth Ng, Toronto (CA); Mojdeh Shakiba, Toronto (CA); Robert Weersink, Toronto (CA); Brian Wilson, Toronto (CA)

(73) Assignee: UNIVERSITY HEALTH NETWORK, Toronto, ON (CA)

(21) Appl. No.: 14/764,403

(22) PCT Filed: Jan. 28, 2014
(86) PCT No.: PCT/CA2014/000062

§ 371 Date: Jul. 29, 2015

Related U.S. Application Data
(60) Provisional application No. 61/757,750, filed on Jan. 29, 2013.

(51) Int. Cl. A61K 49/22; A61K 49/00

(52) U.S. Cl. CPC A61K 49/227; A61K 49/0052; A61K 49/221; A61K 49/0084

(57) ABSTRACT

There is provided herein a nanovesicle having a bilayer comprising a saturated first phospholipid and no more than about 15 molar % of a second phospholipid covalently conjugated to a J-aggregate forming dye.

### RELATED APPLICATIONS

This application claims priority to U.S. Provisional Application No. 61/757,750 filed on, Jan. 29, 2013, which is incorporated herein by reference.

### FIELD OF THE INVENTION

This application relates to nanoparticles and preferably, nanoparticles that J-type aggregates. The application also relates to nanoparticles useful for fluoresence or photo-acoustic imaging or temperature monitoring.

### BACKGROUND OF THE INVENTION

Photoacoustic imaging (PAI) is a novel imaging technique which utilizes the photoacoustic effect as reported by Alexander Graham Bell over 100 years ago (Bell, 1880). This technique, advanced by Kruger (Kruger, 1994; Kruger et al., 1995), Oraevsky (Oraevsky et al., 1997) and Wang (Wang, 2009; Wang and Hu, 2012; Wang et al., 2003) allows for cross-sectional imaging of biological tissues at depths rivaling existing optical techniques. The principles at work in PAT involve the excitation of intrinsic or extrinsic absorbers using a non-ionizing pulsed laser source. Non-radiative relaxation of the excited absorber by vibrational relaxation leads to the generation of acoustic waves which are then detected by an ultrasound transducer. By collecting this acoustic wave using an array of transducers and/or translocation of the detection apparatus, a 3-dimensional image can be generated. PAI is a relatively inexpensive technique and has potential to synergize with other therapies and imaging modalities (i.e. high-intensity frequency ultrasound, photothermal therapy). In particular, intrinsic PAI has been actively investigated as a modality for measuring temperature changes as a result of focal thermal therapy in cancer (Chitnis et al., 2009; Shah et al., 2008). The principle of the technique involves the fact that the measured photoacoustic signal amplitudes depend on the temperature of the source object and the signal amplitudes can be used to monitor the temperature (Pramanik and Wang, 2009). However, there are some limitations as the photoacoustic signal depends on many factors such as the level of coagulation, blood concentration and spectral sensitivity. These factors are in turn affected by biological factors such as the degree of tumor vascularization and tumor size (Esenaliev et al., 1999). Hence, there is a need for a highly sensitive, temperature-dependent PAI contrast agent in which the photoacoustic signal generated will not be sensitive to other uncontrolled and unknown environmental factors.

While monitoring the intrinsic absorbers such as oxy-hemoglobin and deoxy-hemoglobin allows for imaging of the vasculature, tracking exogenous probes allows for the opportunity to monitor molecular processes or to add a layer of functionality to the technique. Exogenous probes tested in conjunction with PAT include small-molecule dyes and metallic nanoparticles; such as, nanoshells, nanorods, nanocages and carbon nanotubes. The large absorption cross-section of metallic nanoparticles in the near-infrared region of the electromagnetic spectrum makes these agents especially suitable for PAI.

It has long been known that organic dyes can self-associate and form molecular aggregates in solution with altered photophysical properties compared with the monomer. Depending on the orientation of packing, J-type or H-type aggregates can be formed. J-type aggregates, also known as J-aggregates, are formed through edge-to-edge packing of the dye molecules and results in narrowing, red-shifting and enhancement of the absorption band. Other properties characteristic of J-aggregation include: a decreased Stokes shift and enhanced fluorescence. These optical properties can be explained by the interaction between Frenkel excitons; electron-hole pairs localized on individual molecules (Knoester, 2003). The shape of the absorption band is affected by the degree of coupling between dyes molecules based upon their intermolecular orientation. As the intermolecular forces facilitating these interactions are weak in nature, J-aggregation is heavily influenced by temperature. At cryogenic temperatures, excitons in certain J-aggregates have been found to be delocalized over 1×10⁷ molecules (Scheblykin, 2012). This is in contrast to the calculated 1000 molecules at room temperature (Scheblykin, 2012).

### SUMMARY OF THE INVENTION

In an aspect, there is provided a nanovesicle having a bilayer comprising (i) a saturated first phospholipid and (ii) no more than about 15 molar % of a second phospholipid covalently conjugated to a J-aggregate forming dye.

In an aspect, there is provided a nanovesicle having a bilayer comprising (i) a saturated first phospholipid and (ii) a second phospholipid covalently conjugated to a J-aggregate forming dye, wherein the dye does not comprise a porphyrin moeity.

In an aspect, there is provided a method of monitoring temperature at a target site comprising: providing the nanovesicle of any one of claims 1-20 at the target site, and monitoring absorbance at the target site; wherein a blue shift in absorbance is indicative of temperature at the target site being higher than a predetermined temperature, the predetermined temperature corresponding to a transition temperature of the saturated first phospholipid, and wherein a red shift in absorbance is indicative of temperature at the target site being lower than the predetermined temperature.

In an aspect, there is provided a method of monitoring temperature at a target site comprising: providing the nanovesicle of any one of claims 1-20 at the target site, and monitoring a photoacoustic signal at the target site; wherein a lack of a photoacoustic signal is indicative of temperature at the target site being higher than a predetermined temperature, the predetermined temperature corresponding to a transition temperature of the saturated first phospholipid, and wherein a presence of a photoacoustic signal is indicative of temperature at the target site being lower than the predetermined temperature.

In an aspect, there is provided a method of monitoring temperature at a target site comprising: providing the nanovesicle of any one of claims 1-20 at the target site, and monitoring a fluorescence signal at the target site; wherein a presence of a blue shifted fluorescence signal is indicative of temperature at the target site being higher than a predetermined temperature, the predetermined temperature corresponding to a transition temperature of the saturated first phospholipid, and wherein a presence of a red-shifted fluorescence signal is indicative of temperature at the target site being lower than the predetermined temperature.

### BRIEF DESCRIPTION OF FIGURES

These and other features of the preferred embodiments of the invention will become more apparent in the following detailed description in which reference is made to the appended drawings, for which a brief description follows.

FIG. 1 shows UV-absorption spectra of Bchl-lipid or Bchl-acid in various lipid environments at 4° C. and 37° C., (A) Absorption spectra of 5% Bchl-lipid in the presence of various phospholipids with either 0 or 1 unsaturated bonds at 4° C. (B) Absorption spectra of 5% Bchl-acid in the presence of various phospholipids with either 0 or 1 unsaturated bonds at 37° C. (C) Absorption spectra of 5% Bchl-lipid in the presence of phospholipids with 0 or 1 unsaturated bonds at 37° C. (D) Absorption spectra of 5% Bchl-acid in the presence of phospholipids with 0 or 1 unsaturated bonds at 37° C.

FIG. 2 shows absorption spectra of varying % mol Bchl-lipid in a saturated lipid environment (with 5% DPPE-PEG2000), showing J-aggregation in formulations containing 5%-50% Bchl-lipid.

FIG. 3 shows structural characterization of J-nanoparticles (A) Negative staining transmission electron micrograph of 15% Bchl-lipid J-nanoparticles and (B) corresponding dynamic light scattering trace.

FIG. 4 shows serum stability experiment of 15% JNPs; in either, PBS or FBS (50%) at 37° C., over a period of 48 hr. Absorption of the J-aggregate peak is measured at 824 nm, normalized to t=0 and expressed as mean +/−standard deviation (n=3).

FIG. 5 shows (A) Photoacoustic image of gel phantom containing Bchl-lipid vesicles in either a DPPC or POPC environment at two wavelengths of interest. When samples are treated with detergent (0.5% Triton X-100) to disrupt the structure, the photoacoustic signal disappears. (B) Corresponding photoacoustic spectra of the samples in A with UV/Vis spectra for comparison.

FIG. 6 shows (A) Temperature melt curve of JNPs prepared with 14-carbon (DMPC), 16-carbon (DPPC), 17-carbon (DHPC), 18-carbon (DSPC) and 19-carbon (DNPC). PA signal was monitored at 824 nm as samples were heated in a waterbath. (B) UV-Visible absorption melt curve of JNPs. (C) UV-Visible absorption melt curve of 15% Bchl-lipid DPPC JNPs showing the reversibility of the JNP's 824 nm absorption peak over multiple heat-cool cycles. (D) Reversibility of 15% Bchl-lipid DPPC JNPs over 5 cycles. Temperature was raised and cooled during each cycle and the signal at 824 nm (green) and 750 nm (red) were recorded. Image of each sample tube during consecutive heat-cool cycles.

FIG. 7 shows temperature response of DPPC JNP loaded into gel phantom during heating (A) PAI of polyacrylamide gel at various times during heating. PA signal at 750 nm (red) and 824 nm (green). (B) Correlation between thermal front (>41° C.) determined from IR and PA.

FIG. 8 shows (A) PA imaging of tumors (n=4) injected with saline (intratumoral; 100 uL) and the influence of heating on PA signal. Image panel on the right shows representative images of the tumor (red scatterplot) at 40° C., 45° C., and 50° C. with top panels showing the ultrasound image (grayscale), blood signal (red; 680 nm-850 nm) and wavelength corresponding to JNPs (green; 824 nm-850 nm). The bottom panels show the 824-850 nm signal alone for clarity. (B) PA imaging of tumors (n=4) injected with 130 uM JNP (intratumoral; 100 uL) and the influence of heating on PA signal. Image panel on the right shows representative images of the tumor (red scatterplot) at 40° C., 45° C., and 50° C. with top panels showing the ultrasound image (grayscale), blood signal (red; 680 nm-850 nm) and wavelength corresponding to JNPs (green; 824 nm-850 nm). The bottom panels show the 824-850 nm signal alone for clarity. (C) PA imaging of tumors (n=3) injected with 130 uM indocyanine green (intratumoral; 100 uL) and the influence of heating on PA signal. Image panel on the right shows representative images of the tumor (red scatterplot) at 40° C., 45° C., and 50° C. with top panels showing the ultrasound image (grayscale), blood signal (red; 680 nm-850 nm) and ICG signal (blue; 810-850 nm). The bottom panels show the 810-850 nm signal alone for clarity.

FIG. 9 is a schematic of hypothesized J-nanoparticle structure below and above transition temperature. Below the transition temperature, Bchl-lipid dyes form J-aggregates with red shift absorption. Above the transition temperature, fluidity in the vesicle membrane inhibits J-aggregation, leading to a recovery of the monomer absorption and a decrease in aggregate absorption.

FIG. 10 shows transmission electron microscope images of JNP ranging from 5-50% Bchl-lipid content. An increase in Bchl-lipid % beyond 15% led to changes in the vesicle morphology. Scale bar represents 500 nm

FIG. 11 shows (A) Absorption spectrum of IRDye QC-1 showing the similar absorbance values at 750 nm and 824 nm. (B) Photoacoustic spectrum showing the similarity of the photoacoustic signal under 750 nm and 824 nm lasing wavelengths.

FIG. 12 shows the difference of internal tumor temperature versus bath temperature during heating experiment. Tissue thermocouple was inserted 2 mm into tumor. Heat rate and water bath mixing velocity was matched to that of experiments in FIG. 4. Temperature differential as measured in each animal during the course of heating. Each bar represents average ±standard deviation of each datapoint in heating trace.

### DETAILED DESCRIPTION

In the following description, numerous specific details are set forth to provide a thorough understanding of the invention. However, it is understood that the invention may be practiced without these specific details.

Many groups have recognized that J-aggregates can be induced in ordered environments such as in polymer films (Zakharova and Chibisov, 2009), DNA (Kawabe and Kato, 2011) phospholipid membranes (Mo and Yip, 2009) and inorganic nanoparticles (Fofang et al., 2011; Walker et al., 2009). Others have also observed that the extent of J-aggregation can be influenced by the physical state of the host environment. In particular, J-aggregation of pseudoisocyanine dyes within a structured phospholipid monolayer can be altered depending on the transition temperature of the host lipid (Mo and Yip, 2009). These dramatic changes in the absorption characteristics are herein harnessed in the development of a smart photoacoustic contrast agent. Applicant describes a J-aggregate forming nanoparticle (JNP) capable of directly responding to environmental changes in temperature with potential application in focal thermal therapy response monitoring.

In an aspect, there is provided a nanovesicle having a bilayer comprising (i) a saturated first phospholipid and (ii) no more than about 15 molar % of a second phospholipid covalently conjugated to a J-aggregate forming dye.

As used herein, “phospholipid” is a lipid having a hydrophilic head group having a phosphate group and hydrophobic lipid tail.

Preferably, the dye is selected from the group consisting of pseudoisocyanine, merocyanine, bis(2,4,6-trihydroxyphenyl)squaraine, Zn-chlorin, tetrtakis(4-sulfonatophenyl)-porphyrin, bacteriochlorin, antimony(III)-phthalocyanine, copper phthalocyanine and perylene bismide, Hypericin, subphtalocyanine, preferably bacteriochlorin. Further preferably, the second phospholipid covalently conjugated to the J-aggregate forming dye is bacteriochlorophyll-lipid.

In another aspect, there is provided a nanovesicle having a bilayer comprising (i) a saturated first phospholipid and (ii) a second phospholipid covalently conjugated to a J-aggregate forming dye, wherein the dye does not comprise a porphyrin moeity. Preferably, the dye is selected from the group consisting of pseudoisocyanine, merocyanine, bis(2,4,6-trihydroxyphenyl)squaraine, Zn-chlorin, antimony(III)-phthalocyanine, copper phthalocyanine and perylene bismide, preferably bacteriochlorophyll.

In some embodiments, the second phospholipid is present in the bilayer in an amount of between 0.01-15 molar %. Preferably, the second phospholipid is present in the bilayer in an amount of between 2-13 molar %.

In some embodiments, the second phospholipid is present in the bilayer in an amount of about 5 molar %.

In some embodiments, the second phospholipid is present in the bilayer in an amount of about 10 molar %.

In some embodiments, the second phospholipid is present in the bilayer in an amount of about 15 molar %.

In some embodiments, the second phospholipid is selected from the group consisting of phosphatidylcholine, phosphatidylethanoloamine, phosphatidylserine, phosphatidylinositol, lyso-phosphatidylcholine, lyso-phosphatidylethanoloamine, lyso-phosphatidylserine and lyso-phosphatidylinositol. Preferably, the second phospholipid comprises an acyl side chain of 12 to 22 carbons.

In some embodiments, the dye is conjugated to the glycerol group on the second phospholipid by a carbon chain linker of 0 to 20 carbons.

In some embodiments, the saturated first phospholipid is selected from the group consisting of phosphatidylcholines, phosphatidylethanolamines, phosphatidic acid, phosphatidylglycerols and combinations thereof. Preferably, the saturated first phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG), 1,2-dipentadecanoyl-sn-glycero-3-phosphocholine (PC(15:0/15:0)), 1,2-diheptadecanoyl-sn-glycero-3-phosphocholine (PC(17:0/17:0)), 1,2-dinonadecanoyl-sn-glycero-3-phosphocholine (PC(19:0/19:0)), 1,2-diarachidoyl-sn-glycero-3-phosphocholine (PC(20:0/20:0)), and combinations thereof.

In some embodiments, the nanovesicle further comprises PEG-lipid. In some embodiments, the nanovesicle further comprises DPPE-PEG2000. In some embodiments, the nanovesicle further comprises DSPE-PEG2000. In some embodiments, the PEG or PEG-lipid is present in an amount of about 5 molar %.

In some embodiments, the nanovesicle is substantially spherical and about 110 nm in diameter.

In an aspect, there is provided a method of monitoring temperature at a target site comprising: providing the nanovesicle of any one of claims 1-20 at the target site, and monitoring absorbance at the target site; wherein a blue shift in absorbance is indicative of temperature at the target site being higher than a predetermined temperature, the predetermined temperature corresponding to a transition temperature of the saturated first phospholipid, and wherein a red shift in absorbance is indicative of temperature at the target site being lower than the predetermined temperature.

In an aspect, there is provided a method of monitoring temperature at a target site comprising: providing the nanovesicle of any one of claims 1-20 at the target site, and monitoring a photoacoustic signal at the target site; wherein a lack of a photoacoustic signal is indicative of temperature at the target site being higher than a predetermined temperature, the predetermined temperature corresponding to a transition temperature of the saturated first phospholipid, and wherein a presence of a photoacoustic signal is indicative of temperature at the target site being lower than the predetermined temperature.

In an aspect, there is provided a method of monitoring temperature at a target site comprising: providing the nanovesicle of any one of claims 1-20 at the target site, and monitoring a fluorescence signal at the target site; wherein a presence of a blue shifted fluorescence signal is indicative of temperature at the target site being higher than a predetermined temperature, the predetermined temperature corresponding to a transition temperature of the saturated first phospholipid, and wherein a presence of a red-shifted fluorescence signal is indicative of temperature at the target site being lower than the predetermined temperature.

Advantages of the present invention are further illustrated by the following examples. The examples and their particular details set forth herein are presented for illustration only and should not be construed as a limitation on the claims of the present invention.

### Examples

### Methods and Materials

### Materials

All phospholipids were purchased from Avanti Polar Lipids Inc. (Alabaster, Ala.) and reconstituted with chloroform prior to utilization. Bacteriochlorophyll-conjugated lipid was synthesized as previously reported (Lovell et al., 2011). Polyethylene tubing with (1.09 mm internal diameter) was purchased from Becton Dickinson and Company (Sparks, Md.) and was thoroughly washed with ethanol before use. Agarose was purchased from BioRad (Mississauga, ON), while fetal bovine serum was purchased from Wisent (St. Bruno, QC). Extruder drain discs and polycarbonate membranes were purchased from Whatman (Piscataway, N.J.)

### J-Nanoparticle Synthesis

JNPs were made by the lipid extrusion technique as previously described. Briefly, Bchl-lipid, PEG2000-DPPE and host lipids dissolved in chloroform were transferred to borosilicate glass tubes and dried by N₂ to form a thin film (Table 1-1). For serum stability studies, cholesterol (40 mol %) was added to the formulation. This film was then transferred to a vacuum desiccator and dried for an additional 30 min to ensure complete solvent removal. Films were hydrated with PBS and subjected to 5 freeze-thaw cycles and extruded through two 100 nm polycarbonate membranes using a hand extruder or a high pressure extruder set to a temperature of 65° C. Prepared samples were transferred to 1.5 mL Eppendorf tubes and stored at 4° C. until used. For the study investigating the role of phospholipid structure on J-aggregate formation, the sonication method was used to synthesize the JNPs. Briefly, films dried as described previously were subjected to 5 freeze-thaw cycles and sonicated at 65° C. for 1 hr in a temperature controlled bath sonicator until the solution was clarified.

### J-Nanoparticle Spectral Characterization

The UV/Vis absorption ratio of J-nanoparticles was measured in PBS using a Varian Cary 50 UV-visible spectrophotometer (company and country). This measurement was divided by the number of moles of Bchl-lipid (37 000M⁻¹cm⁻¹; 1100 MW) in the solution to estimate the molar extinction coefficient of the aggregated molecule.

### Transmission Electron Microscopy

Transmission electron microscopy was carried out on a Hitachi H-7000 electron microscope with an acceleration voltage of 75 kV. Ten microliters of sample was applied to a glow discharged 200-mesh copper-coated grid. The sample was washed with ddH₂O and stained with 2% uranyl acetate.

### Serum Stability Studies

For serum stability studies, JNPs were incubated with 0% and 50% fetal bovine serum at 37° C. over 48 hours. Time points measured include: 0, 0.5, 1, 6, 24 and 48 hr time points. At each time point, sample was withdrawn from the incubation tube, transferred to 96-wellplate and the absorbance measured at 824 nm.

### Photoacoustic Imaging

Photoacoustic imaging was performed using a Vevo 2100 LAZR photoacoustic imaging system (Visualsonics, Toronto, ON) equipped with a 21 MHz-centered transducer and a flashlamp pumped 20 Hz Q-switched Nd-YAG laser, tunable from 680-970 nm with a 1 nm step size. The gel phantoms were prepared by pouring 60 mL a boiling 1% agarose solution into a 10 cm Petri dish. Once slightly cooled, an electrophoresis gel comb was placed in the gel and allowed to solidify. The comb was then removed and the wells were filled with the sample mixed with agarose (0.5% final).

### Absorbance-Temperature Profiles

Absorbance-temperature profiles were collected on the temperature controlled Jasco J-815 CD spectrophotometer. Measurements were subtracted from baseline measurements using PBS at 824 nm. A metal thermocouple was inserted into the cuvette for monitoring temperature. The temperature within the sample cell was gradually heated from 20-60° C. at 5° C. min⁻¹ with absorbance measurements made every 0.1° C. Temperature cycling experiments were conducted as described above with ramp temperatures set from 25-60° C. at a rate of 5° C. min⁻¹. The same temperature gradient was set for the cooling step.

### Photoacoustic Signal-Temperature Profiles

Photoacoustic imaging was performed using a Vevo 2100 LAZR photoacoustic imaging system (Fujifilm, Toronto, ON) equipped with a 21 MHz-centered transducer and a flashlamp pumped 20 Hz Q-switched Nd-YAG laser, tunable from 680-970 nm with a 2 nm step size. Photoacoustic-temperature profiles were collected in a custom-built heating apparatus comprised of 5 polyethylene tubing fixed within a plastic holder. The plastic tubing and holder was submerged in a glass beaker filled with degassed water and a stir bar. Tubes in the heating apparatus were loaded with JNPs prepared with host phospholipids of various acyl chain lengths. The photoacoustic transducer was placed such that the ultrasound array captured an image slice through each tube. The temperature in the bath was increased from 25-60° C. using a hot plate while being monitored using a thermocouple placed in the same depth of water as the plastic tubing.

### Photoacoustic Imaging in Gel Phantoms

Polyacrylamide photoacoustic hydrogel phantoms were prepared using the method described by Choi and colleagues with modification. Briefly, 59.06 mL ddH2O, 30 mL of 30% (w/v) 19:1 acrylamide and 10 mL of 1M Tris buffer (pH 8) were combined in an Erlenmeyer flask and degassed under vacuum for 15 min. Ammonium persulfate (APS; 10% w/v) and N,N,N′,N′-tetramethylethylenediamine (TEMED) were added to the monomer solution such that the final concentration was 0.84% and 0.2%, respectively. Polymerizing solution was rapidly poured into a custom built rectangular gel mold and comb and allowed to polymerize for 1 hr. The monomer solution was prepared once again, however a volume of the ddH2O was replaced with a solution of JNP such that the final JNP concentration was 30 μM. Once the outer gel was polymerized, the comb was removed and the empty space was filled with the newly prepared JNP gel solution. Gels were used immediately after polymerization.

Photoacoustic imaging was performed using a Vevo 2100 LAZR photoacoustic imaging system (Fujifilm, Toronto, ON) equipped with a flashlamp pumped 20 Hz Q-switched Nd-YAG laser, tunable from 680-970 nm with a 2 nm step size. Hydrogel phantoms were placed on a resistive heating element (20V; 25 cm2; McMaster-Carr; cat#35475K263) to provide heat to the JNP filled gel. PA images were collected on the gel phantom during the experiment by aligning a 21 MHz transducer array parallel to the direction of heating and scanning across the gel to generate a 3D image of the gel. The excitation wavelength was alternated between 750 nm and 824 nm during the scan. While the gel images were scanning, thermographic images were captured using an IR camera placed perpendicular to the direction of heating.

Thermographic images were analyzed using the Mikrospec™ 4.0 imaging software. All other analysis was conducted using ImageJ. The thermal front exceeding 41° C. in the hydrogel phantom was measured and compared with the thermal front determined from the decrease in signal intensity at 824 nm. The data was fit using linear least squares regression through the origin.

### Measurement of Photoacoustic Signal-Temperature Profiles in Tumor Xenografts

All animal procedures were approved and conducted in accordance with University Health Network's Animal Research Committee. KB cells were cultured in Eagles Minimum Essential Medium supplemented with 10% fetal bovine serum. Immediately prior to tumor inoculation, KB cells were trypsinized and washed 3 times with phosphate buffered saline. The concentration of cells was adjusted to 2×10⁷ cells/mL and kept on ice throughout the experiment. Animals were anaesthetized with a gaseous mixture of isofluorane and oxygen. Once induction of anaesthesia was complete, the hind flank of each animal was inoculated with 2×10⁶ cells.

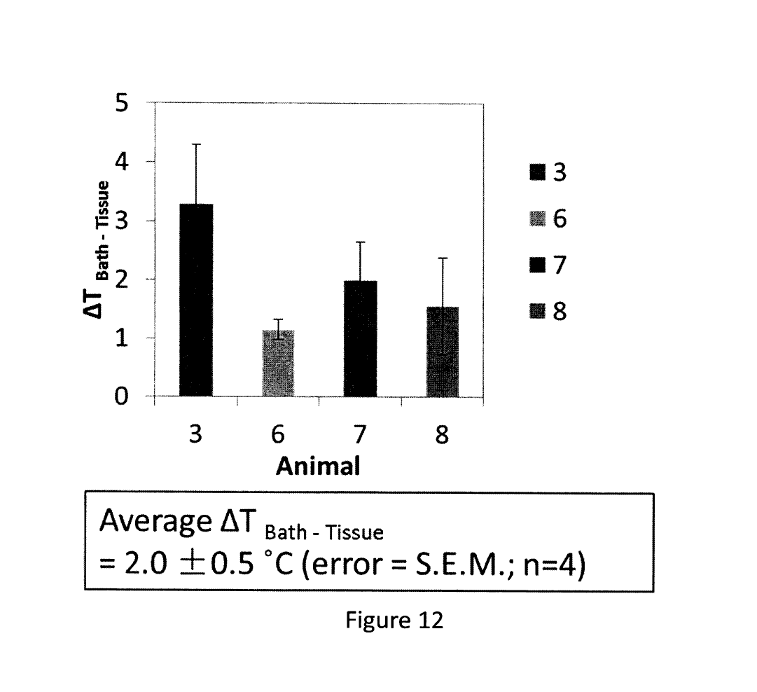

Heating experiments to test the effect of heating on the signal change of JNPs once tumors reached an appropriate size (average volume=263 mm³). Anaesthetized animals were placed on a stage in the PA imager. The hindlimb was immobilized in a temperature controlled waterbath (FIG. 12). The temperature in the bath was slowly raised during the heating procedure (average heating rate=1.8° C./min) and monitored using a thermocouple. Animals were split into three groups each receiving 100 uL of saline (n=4), DPPC JNP (130 μM; n=4) or indocyanine greern (130 μM; n=4) delivered through a 21G needle inserted 2 mm below the surface of the tumor. Immediately after injection, a 21 MHz PA transducer was placed on the tumor and images were collected throughout the heating procedure. The water bath temperature was increased from 25-50° C. while the ultrasound and PA images were collected. For PA imaging, the excitation laser wavelength was cycled sequentially between 680 nm, 750 nm, 800 nm, 824 nm and 850 nm. To determine the extent of a temperature differential in the waterbath versus within the tumor, thermocouples were inserted in the waterbath as well as within the KB tumor. The tip of the thermocouple was buried 2 mm below the surface of the tumor. Heating on the tumors were conducted as described above (vide supra). The bath temperature and the tissue temperature were compared and the difference between the two calculated for each temperature point.

To quantify the PA signal arising from the JNPs as a function of temperature, regions of interests were drawn around the center of the tumor for each animal. The signal at each of the 5 wavelengths was plotted versus bath temperature. This was determined by matching the PA image time with the thermocouple measurement time. The PA signal at 850 nm was used as the baseline throughout the experiment as the absorption from endogenous absorbers was minimal. All other wavelengths were subtracted from this wavelength derive the corrected PA signal. For the temperature versus PA signal plots, all data were normalized to the maximum value of the trace

TABLE 1-1
List of lipids used for screen of J-aggregation conditions

| | | | Number of | |
|---|---|---|---|---|
| Structure of | | | unsaturated | Lipid transition |
| Bchl | Host lipid | Abbreviation | bonds | temperature (° C.) |
| Bchl-lipid | 1,2-dielaidoyl-sn-glycero-3-phosphocholine | 18:1 Trans PC | 1 | 12 |
| | 1,2-dioleoyl-sn-glycero-3-phosphocholine | 18:1 Cis PC | 1 | −20 |
| | 1,2-dipalmitelaidoyl-sn-glycero-3-phosphocholine | 16:1 Trans PC | 1 | — |
| | 1,2-dipalmitoleoyl-sn-glycero-3-phosphocholine | 16:1 Cis PC | 1 | −36 |
| | 1-palmitoyl-2-oleoyl-sn-glycero-3-phosphocholine | POPC | 1 | −2 |
| | 1,2-dipalmitoyl-sn-glycero-3-phosphocholine | DPPC | 0 | 41 |
| | 1,2-dimyristoyl-sn-glycero-3-phosphocholine | DMPC | 0 | 23 |
| Bchl-acid | 1,2-dielaidoyl-sn-glycero-3-phosphocholine | 18:1 Trans PC | 1 | 12 |
| | 1,2-dioleoyl-sn-glycero-3-phosphocholine | 18:1 Cis PC | 1 | −20 |
| | 1,2-dipalmitelaidoyl-sn-glycero-3-phosphocholine | 16:1 Trans PC | 1 | — |
| | 1,2-dipalmitoleoyl-sn-glycero-3-phosphocholine | 16:1 Cis PC | 1 | −36 |
| | 1-palmitoyl-2-oleoyl-sn-glycero-3-phosphocholine | POPC | 1 | −2 |
| | 1,2-dipalmitoyl-sn-glycero-3-phosphocholine | DPPC | 0 | 41 |
| | 1,2-dimyristoyl-sn-glycero-3-phosphocholine | DMPC | 0 | 23 |

TABLE 2-1
Composition of J-nanoparticles to study the influence of Bchl-lipid
content on optical spectra

| | | | PEG2000- |
|---|---|---|---|
| Bchl-lipid (%) | Host lipid | Host lipid (%) | DPPE (%) |
| 5 | DPPC | 90 | 5 |
| 10 | DPPC | 85 | 5 |
| 15 | DPPC | 80 | 5 |
| 20 | DPPC | 75 | 5 |
| 50 | DPPC | 45 | 5 |

### Results and Discussion

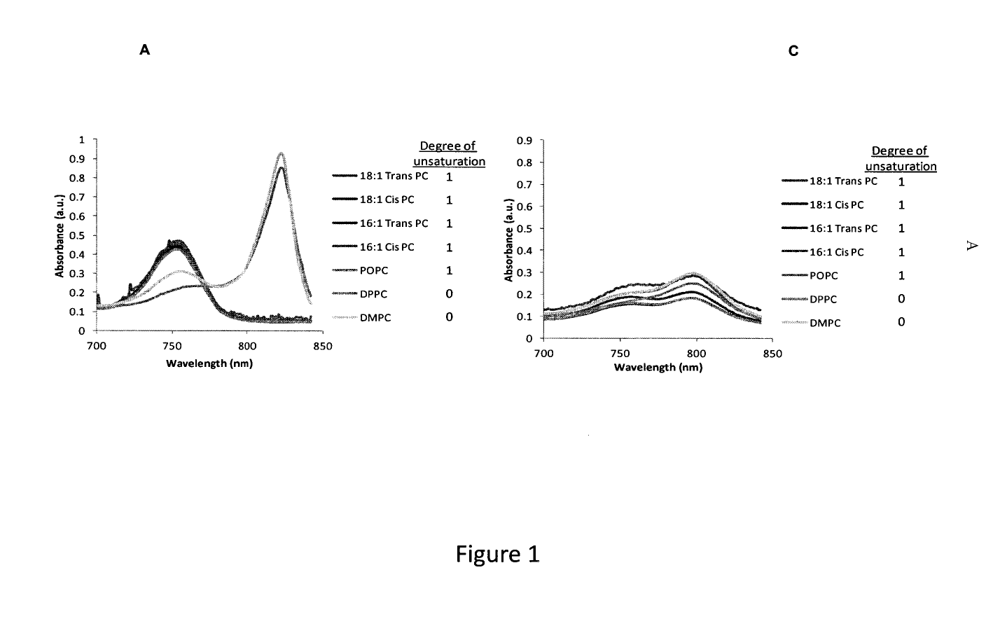

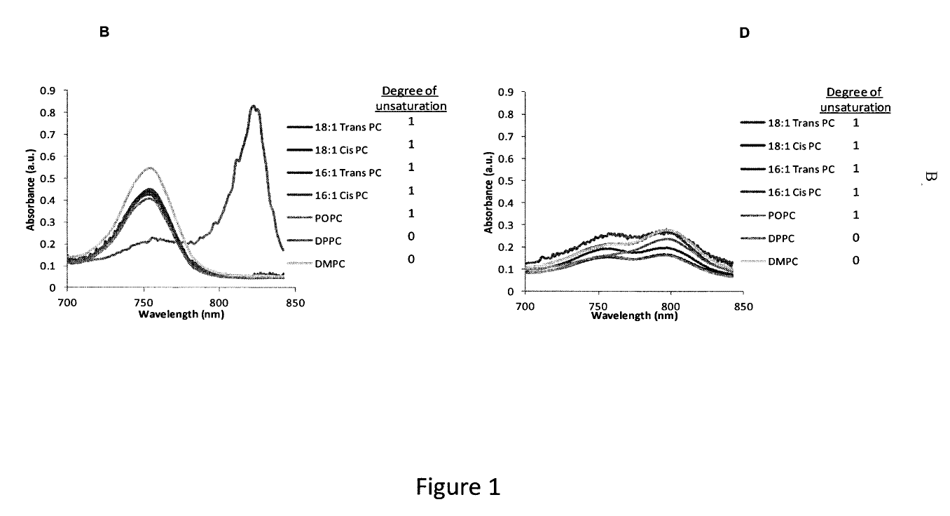

Previous studies have examined the influence of host lipid structure on the formation of J-aggregates in pseudoisocyanine (PIC) dyes. Applicants initiated our study by examining the J-aggregating propensity of two modified bacteriochlorophyll analogs. Applicants chose a parent bacteriochlorin structure for our studies as these molecules are important in photosynthesis for a number of bacterial species and have been shown to form Frenkel type excitons in the bacterial light harvesting apparatus. Specifically, the two exemplary structures applicants examined were a bacteriochlorin with a carboxylic acid (Bchl-acid) and the other with a phosopholipid conjugation (Bchl-lipid). In one example, Applicants fixed the composition of each formulation at 5 mol % Bchl and 95 mol % host lipid. For both Bchl-acid and Bchl-lipid, applicants prepared a series of formulations with a series of lipids with variations in the chemical structure as well as the phase transition temperature (Table 1). Each prepared film was hydrated with PBS and sonicated at 65° C. for 1 hr. Samples were next adjusted to the same Bchl concentration and transferred to a 96-wellplate. Wavelength scans from 700-850 nm were made using a temperature-controlled plate reader. Measurements made of Bchl-lipid containing samples at 4° C. showed that host lipids containing an unsaturated bond caused Bchl-lipid to absorb at the monomer absorption band (750 nm) (FIG. 1A). In contrast, red-shifted peaks were observed in formulations containing saturated lipids. This peak at 824 nm was more intense and narrow as compared with the monomer band. When the temperature of the formulations was raised to 37° C., similar trends were observed in samples with unsaturated bonds in the host lipid. However, in the formulation prepared with DMPC, a drastic change in the spectra was observed (FIG. 1B). The aggregate absorption band disappeared and was replaced by a single peak centered around the monomer absorption band. The longer acyl chain length formulation prepared with DPPC showed no changes with exception of a slight decrease in the intensity of absorption. When examining the influence of host lipid composition on the spectra of Bchl-acid at 4° C., applicants observed negligible differences between the absorbance spectra of all formulations (FIG. 1C). Furthermore, this trend was also observed when the temperature was elevated to 37° C. (FIG. 1D).

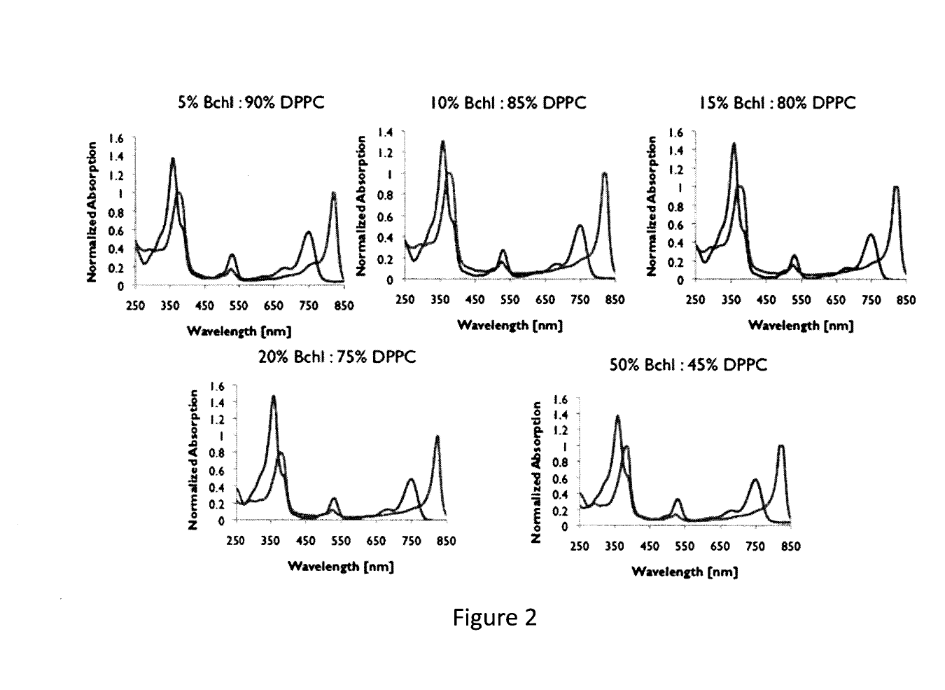

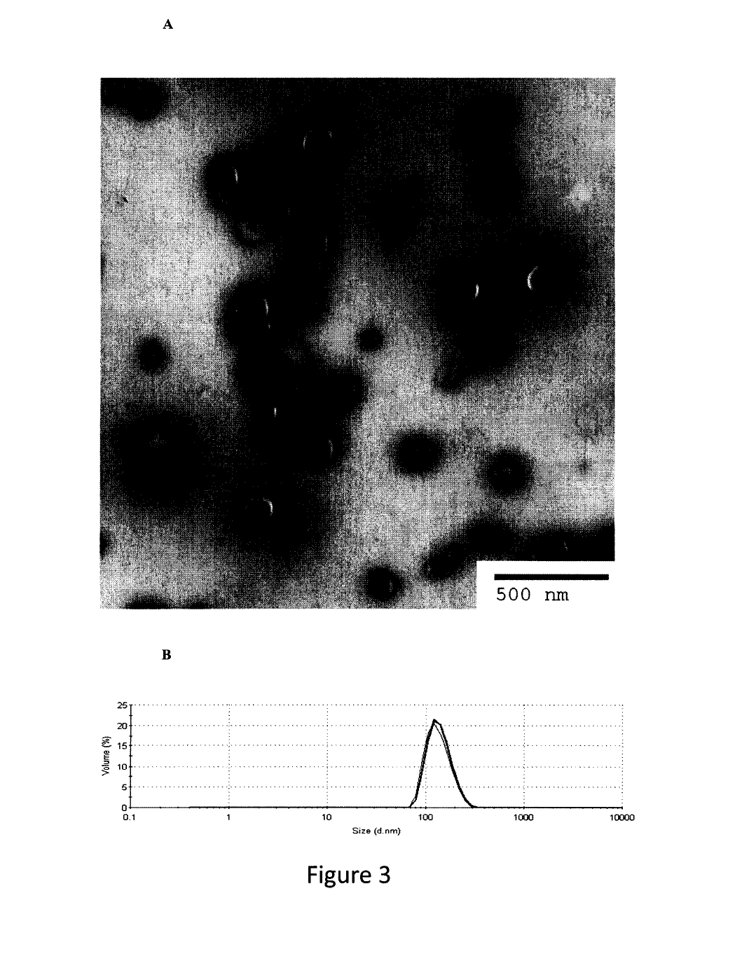

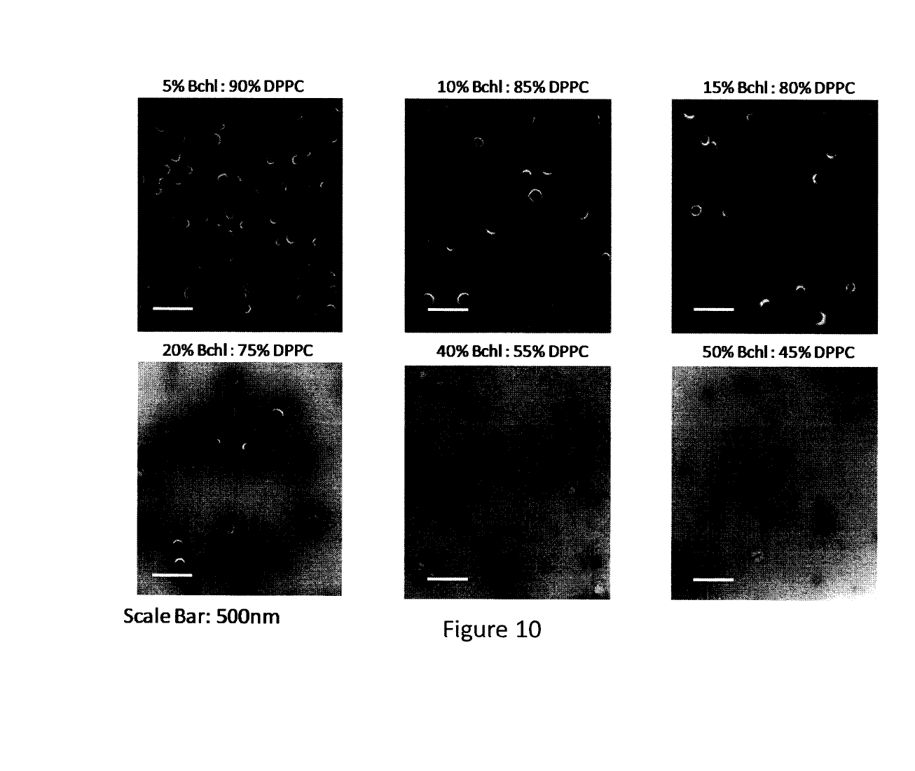

Applicants next examined the influence of % Bchl-lipid on the observed spectra of the JNPs. Applicants began the experiments by varying the % Bchl-lipid from the initial 5% up to a maximum of 50%. These formulations all contained 5% PEG2000-DPPE as applicants were interested in generating JNPs with favorable in vivo stability properties. Applicants chose DPPC as the host lipid as it had a transition temperature that was conducive for measurements at room temperature. Applicants also prepared these lipid vesicles using the extrusion technique as it allows for greater monodispersity. Once these samples were prepared, absorbance spectra were measured at room temperature (FIG. 2). Applicants found the composition of the formulation had no effect on the spectral shift. However, the extinction coefficient of the peak appeared to fluctuate when normalized to the Bchl concentration in the sample. There was no discernable trend observed over the range of 5-50% Bchl-lipid (mol/mol). However, in all cases the extinction coefficient exceeded that of the monomer solution. These formulations were kept at 4° C. over several days to determine their storage stability. In addition, negative staining transmission electron microscope images were captured of each formulation over the course of 1 week. Applicants found that when the Bchl-lipid percentage was increased beyond 15%, changes in the JNP morphology were observed (FIG. 10). However, when applicants kept the percentage of Bchl-lipid at 15% or lower, only spherical vesicle shapes with a volume mean of 110 nm was observed (FIG. 3).

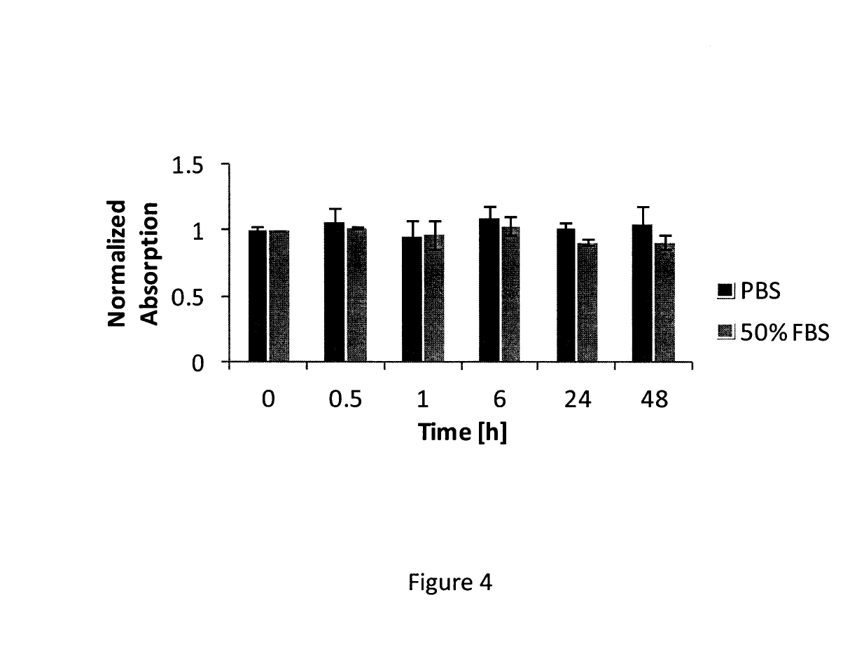

Serum stability experiments were conducted to assess the effect of serum lipids and proteins on the stability of the J-aggregate absorption band (FIG. 4). Fifteen percent Bchl-lipid JNPs was incubated with either 50% fetal bovine serum or PBS and monitored over a period of 48 hr at 37° C. No statistical differences were observed over the 48 hr experiment

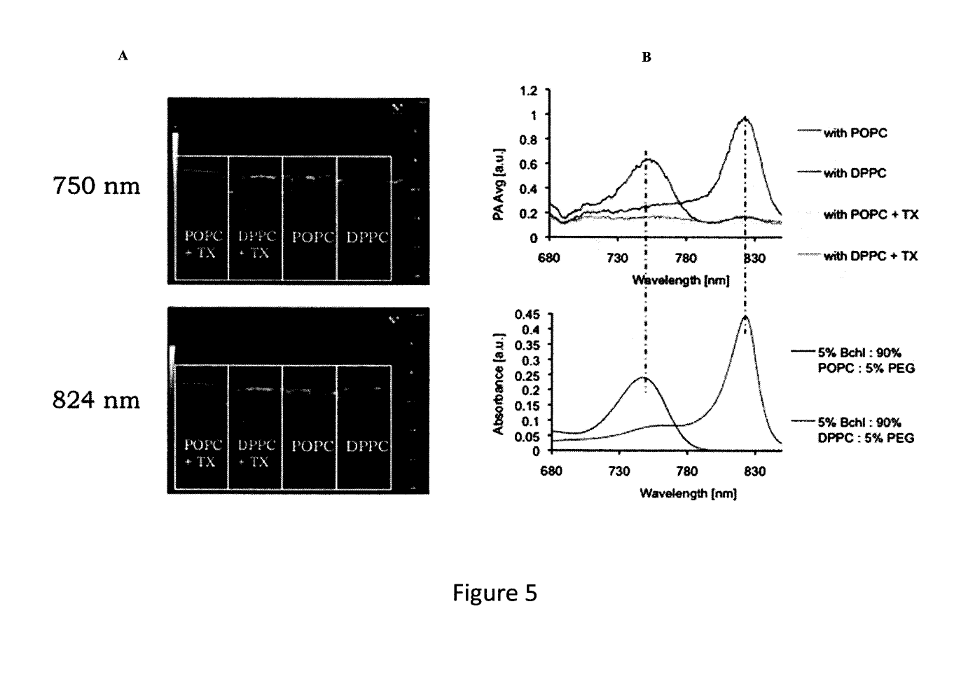

Applicants next proceeded to determine whether applicants could observe a photoacoustic signal when the JNPs were injected into an agarose gel phantom. 50 uM of 15% Bchl-lipid JNPs made using a J-aggregating, a non-J-aggregating host lipid were injected into the agarose gel phantom. These two samples were also mixed with triton x-100 as controls to determine if the photoacoustic signal from the monomer could be observed (FIG. 5A). Photoacoustic images of the agarose phantom cross-sections were collected over wavelength range of 680-850 nm (FIG. 5B). Large signal intensities were observed at 824 nm for the JNP prepared with DPPC. Interestingly, in the non-J-aggregating sample, the presence of the unsaturated lipid shifted the maximal photoacoustic signal to 750 nm. The observed photoacoustic signal peaks overlaid well with the UV/visible spectroscopy data.

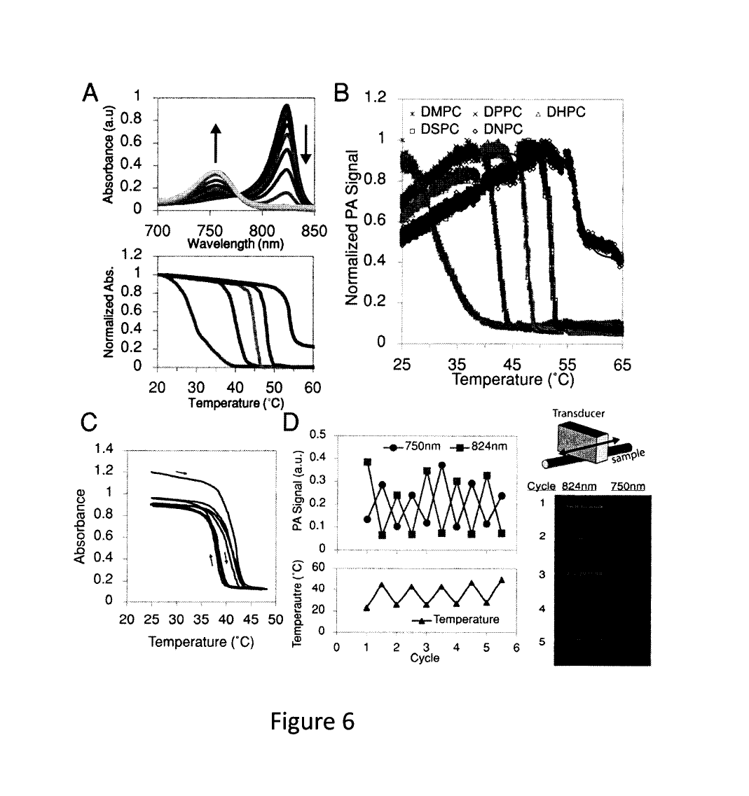

As illustrated in previous experiments, there appeared to be a temperature dependent change in the absorption properties of the JNPs. To confirm and further expand on these results, applicants conducted a thorough examination on the effect of adding Bchl-lipid at 15 mol % to a variety of host phospholipids. Applicants examined phospholipids with acyl chain lengths of 14 (DMPC), 16 (DPPC), 17 (DHPC), 18 (DSPC) and 19 (DNPC) carbons and their effects on the absorbance change at 824 in response to heat. As the temperature increased in each sample, the signal decreased at a temperature near the phase transition temperature midpoint of the phospholipid (FIG. 6A). While a change in absorbance was observed, applicants repeated the experiment but monitored the PA signal in response to temperature. Similar to the trend previously observed with the absorbance melt curves, the PA signal decreased with increase temperature with the midpoint varying based on the phase transition temperature of the host lipid (FIG. 6B).

Another observation made of our nanoparticle system was that the structural changes responsible for causing disruption of J-aggregates was reversible. To clarify the nature of this interaction, applicants conducted multiple heating-cooling cycles on our JNPs. In the case of JNP prepared using DPPC, the phase transition temperature midpoint remained the same during heating, thus showing the robustness of the system. This was in contrast to the cooling step, which resulted in a phase transition temperature midpoint which was on average 3° C. lower than observed during heating (FIG. 6C). This observed temperature hysteresis could possibly be explained by the required relaxation time for the monomer to reassemble into J-aggregated domains. Applicants next demonstrated the reversible measurement of temperature by PA imaging. Polyethylene tubes were filled with a solution of DPPC JNPs and sequential heat-cool cycles were conducted. Measurements were made either below or above the midpoint of the phase transition temperature. Repeated ramping of the temperature led to repeated cycling of the PA signal at 824 nm (FIG. 6D). The decrease of the signal at 824 nm coincided with an increase of the monomeric PA signal at 750 nm. Taken together, this data suggests that the sensing threshold can be tuned by varying the lipid composition and sensing can be reversibly achieved over several heat-cool cycles.

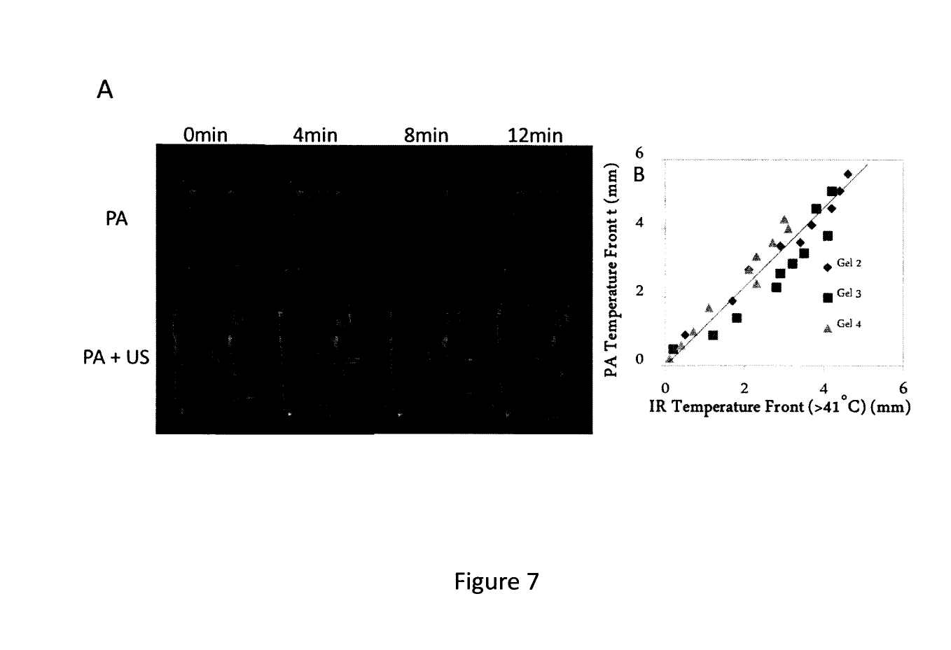

To prove that this nanoparticle can be used to spatially track a thermal front, JNPs prepared with DPPC (14-carbon) were embedded in a polyacrylamide gel phantom which mimics the ultrasound properties of tissue. Gels were heated from one face using a resistive heating element while PA and infrared images were collected at an angle perpendicular to the direction of heating. Scans across the gel surface were captured at various points during heating and reconstructed showing the PA signal at 750 nm and 824 nm (FIG. 7A). During the course of heating, the progression of the temperature front could be observed by a wave of diminishing signal at 824 nm. This signal decrease was confirmed to be the disaggregation of the J-aggregate peak, not as movement of the nanoparticle out of the imaging view since an increase in the monomeric PA signal at 750 nm was observed. Images collected by the PA transducer were compared with temperature measurements made using a thermographic camera by measuring the length of the thermal front exceeding 41° C. in infrared images. The temperature (41° C.) front determined by thermographic imaging was found to be well correlated with the thermal front determined by PA imaging (R2=0.92) (FIG. 7B).

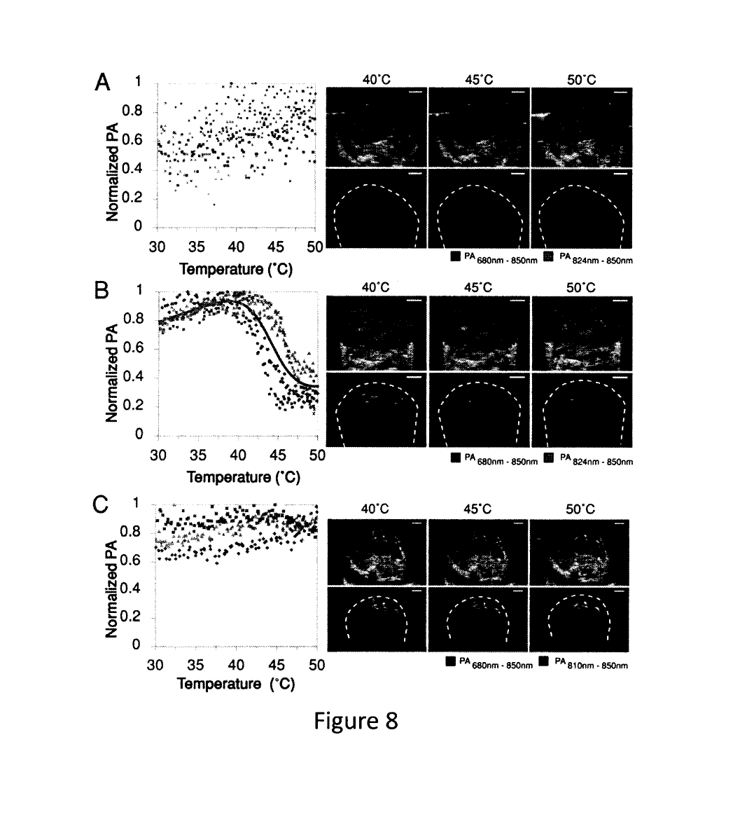

Once it was determined that this technique can be used to monitor the progression of a temperature front, applicants next sought to observe this trend in vivo where tissue composition can affect the signal captured by PA imaging. Applicants explored the possibility of using JNP-based PA temperature sensing in murine tumor xenografts. KB-tumor bearing mice KB tumors were seeded in the hind flanks of each animal prior to the start of the heating experiment. Animals were anaesthetized and immobilized in a custom-built waterbath. Animals were intratumorally-injected with 100 μL of saline, DPPC JNP (130 μM) or indocyanine green (ICG; 130 μM) through the intratumoral route and immobilized in a custom-built waterbath. The temperature in the bath was increased from 25-52° C., while a PA transducer collected images at various wavelengths (680 nm; 750 nm; 800 nm; 824 nm; 850 nm). Region of interests were drawn over tumors and the PA signal was plotted versus temperature. In the case of these traces, each value was normalized to max. Animals injected with saline did not display any enhancement in signal at 824 nm, while blood in the tumor can be visibly observed at 680 nm (FIG. 8A). Animals injected with 130 μM DPPC JNP showed clear signals originating from the center of the tumor at 824 nm (FIG. 8B). This signal can be observed over the blood owing to the reduced optical absorption within the near infrared wavelength range. Heating the tumor in the bath resulted in an initial increase in the J-aggregate signal, followed by a dramatic decrease in the signal. The midpoint of this observed transition was 44° C. This PA signal profile was different from animals receiving an injection of ICG indicating that the change in signal did not arise due to imaging artifacts such as motion. While a signal increase was observed, the signal did not disappear upon exceeding bath temperatures of 40° C. Based on the comparison with the saline and ICG injection, it was determined that JNPs could be used with PA to monitoring temperature changes within a biological environment. The discrepancy between the measured phase transition midpoint within the tumor versus the in vitro measurements could be potentially be explained by a temperature lag between the water bath and the intratumor environment caused by tissue insulation and tumor blood flow. To test this hypothesis, controlled studies were conducted to determine the extent of the temperature lag. Thermocouples were inserted in both the waterbath and the hind limb tumor of animals. Thermocouples were inserted 2 mm into the center of the tumor and differences between external and internal environment were measured (FIG. 12). The average temperature differential was 2.0±0.5° C., which indicated that the measured midpoint of the JNP phase change in the tumor was on average approximately 2 centigrade higher than the internal tumor temperature at 2 mm below the skin surface.

PAI, with its advantages over other optical techniques has garnered attention for its unparalleled signal depth resolution and its ability to image endogenous process by exciting endogenous absorbers. Its companion approach, contrast-enhanced PAI is an active field of research, as it can provide additional information into biological processes in healthy and disease states, especially when coupled with an appropriate targeting moiety. In particular, nanoparticle-based contrast agents greatly extends the utility of PAI as they can potentially encapsulate large numbers of imaging dyes per nanoparticle (Kim et al., 2007; Lovell et al., 2011) and in the case of metallic nanoparticles, can utilize the nanoscale property of surface plasmon resonance to tune and greatly enhance the absorption coefficient of the nanoparticle.

One nanoscale property which has yet to be investigated in PAI is the phenomenon of J-aggregation in organic dyes. J-aggregation causes a red-shift, narrowing and enhancement of the dyes absorption band. In addition, the reversible, weak, intermolecular interactions governing the association of J-aggregating dye molecules provide a unique mechanism which can be harnessed to create sensors responsive to the local environment of the dye. As all of these effects synergize well with PAI, applicants were interested to see how applicants could utilize the properties to create a smart nanoparticle-based PAI contrast agent.

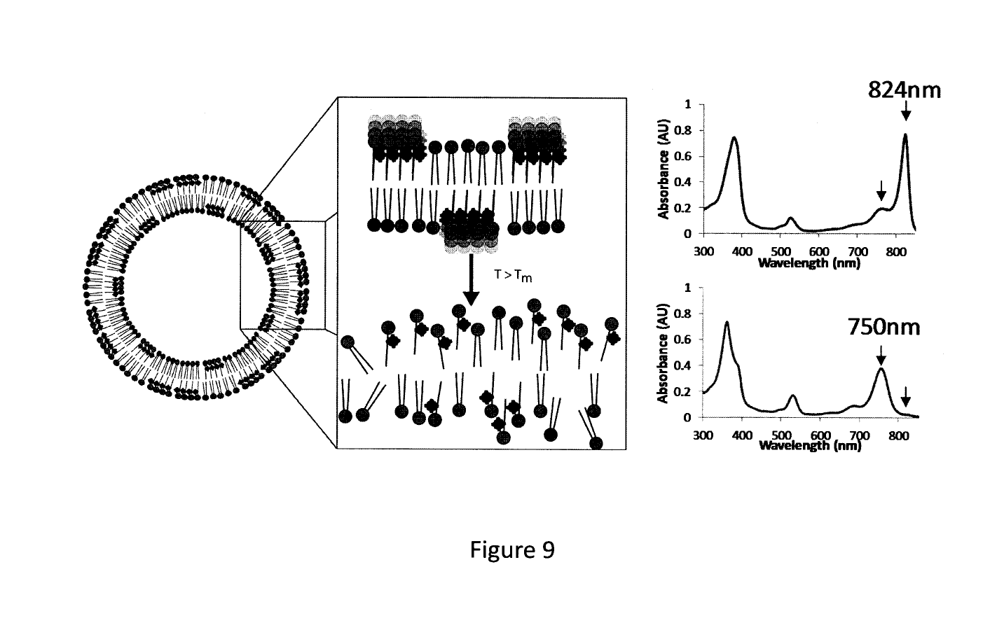

Applicants began our investigation by examining the J-aggregating potential of two dyes based on the bacteriochlorin parent structure. Applicants chose to focus our work on bacteriochlorin as it has a monomer absorption maximum at 750 nm in the near-infrared range. Furthermore, J-aggregation in this class of molecules have previously been reported in the chlorosomes of photosynthetic bacteria (Prokhorenko et al., 2003). Applicants began our study with a lipid screen comparing Bchl-lipid in a series of host phospholipids. Applicants hypothesized that the structure of the conjugated dye as well as the structure of the host lipid will influence whether J-aggregation occurs. When comparing the phospholipid-conjugated bacteriochlorophyll with the unconjugated analog, applicants observed that J-aggregation only occurred in the lipid conjugated compound (FIG. 1). Conjugation of the dye to the phospholipid likely influences J-aggregation as it limits the number of possible orientations the dye can adopt in the membrane so as to ease the interaction with other dyes. When examining the influence of the host lipid, applicants observed that at 4° C., J-aggregation only occurred with the saturated phospholipids. However, when the temperature was elevated to 37° C., surprisingly the formulation with the DMPC host lipid showed a conversion of the J-peak back to a monomer peak. The temperature range in which this was observed coincided with the phase transition temperature of DMPC. As such an explanation of the observed phenomenon may involve lipid disorder having inhibitory effects on J-aggregation (FIG. 9).

Applicants next studied whether the percentage loading of Bchl-lipid influenced J-aggregation. Applicants found that from 5-50% Bchl-lipid, no significant changes in the J-aggregate spectrum could be observed. In all cases, the measured molar extinction coefficient exceeded 80 000 M⁻¹cm⁻¹. However, when Bchl-lipid % was increased beyond 15%, changes in size and shape of the nanoparticle occurred after storage for several days at 4° C. Interestingly, even though the particle was not structurally stable, the J-aggregate spectra was maintained. Characterization of the 15% nanoparticle formulation showed that these nanoparticles had a vesicular structure consistent with the shape of liposomes and were approximately 110 nm in size. The lack of dependence on structure implies that dye domains involved in J-aggregation was small enough to not be affected by the curvature of the JNP.

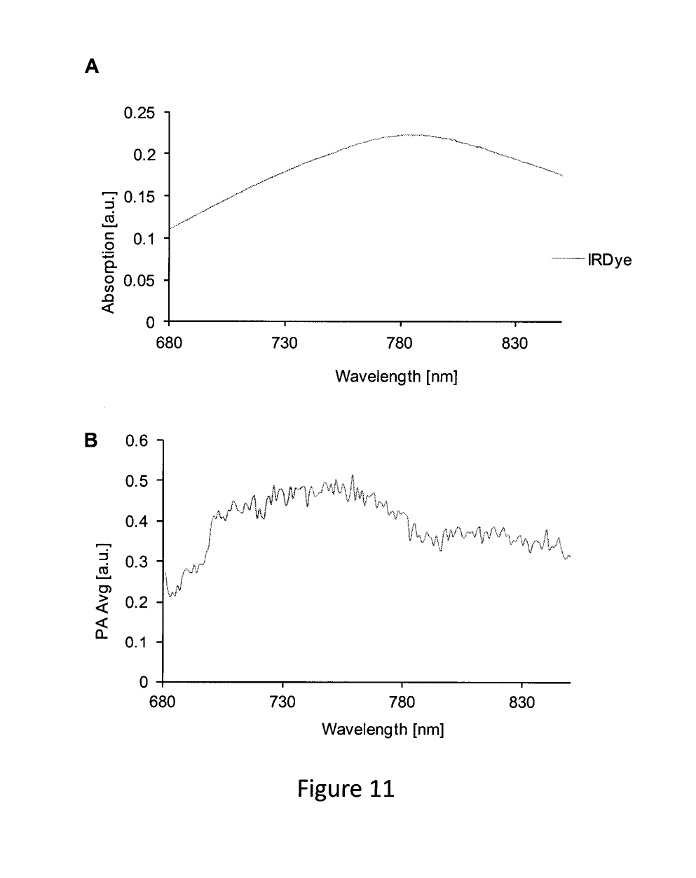

The application of J-aggregation as a mechanism to enhance the PAI using organic dyes has not previously been demonstrated. The photoacoustic signal of two JNP formulations in an agarose gel phantom was examined (FIG. 5). Similar to the trend observed by UV/Vis spectroscopy, applicants found that the J-aggregating JNP prepared with DPPC displayed a greater signal intensity over the control nanoparticle made using POPC (a non-J-aggregating environment). To account for any wavelength-dependent differences in the laser excitation, applicants ran a control using a solution of IRDye QC-1 which had uniform absorbance and photoacoustic response within the wavelengths of interest (FIG. 11). Applicants found equivalent photoacoustic signal emanating from the dye at the 750 nm and 824 nm wavelengths demonstrating that the behavior of the apparatus behaved similarly in the region applicants were interested in.

Another objective was to determine whether the temperature dependence of J-aggregation can be used as a mechanism to sense changes in the local environment around JNP. By inserting Bchl-lipid into a host membrane with various transition temperatures, applicants hoped to modulate the temperature at which the J-aggregate signal was lost. The most direct to test this was to load the various formulations into a tube phantom in a water bath. The temperature of the bath was varied and the photoacoustic signal was scanned. When applicants plotted the signal intensity of the J-band with temperature, applicants observed that the signal generally varied with the transition temperature of the lipids tested. However, DPPC appeared to show the sharpest decrease around its transition temperature. DSPC also showed a temperature-dependent decrease but the drop was not as dramatic as that of DPPC. Further experiments will determine whether this can be improved. It is also noted that the time it took for the PA signal change to occur was on the ×10⁰ seconds timescale.

Applicant also examined the capability of using this technique to monitor temperature in tissues during heating (FIG. 8). Tumor-bearing mice injected with JNPs showed a decrease in PA signal in response to thermal increases beyond the midpoint of the host lipid's phase transition temperature. Similar controls injected with saline or an equivalent concentration of organic dye (indocyanine green) did not display a decrease in PA signal in response to heating providing further evidence of temperature sensing in the JNP system.

The experiments conducted herein demonstrate the possibility of forming J-aggregates in a variety of lipid environments. Furthermore the environment that the lipid is in can dictate the spectral properties of the dye. Applicants also showed that J-aggregation can be used to enhance the photoacoustic signal of J-aggregating organic dyes and that at least in the case of Bchl-lipid, this change represents an improvement, as the spectra becomes red-shifted further into the tissue optical window with a concomitant increase in signal intensity. Also, applicants have shown that JNPs can potentially be used in monitoring temperature of various focal thermal therapies as the J-aggregate induced PAI contrast enhancement is temperature dependent. We've shown that JNP can potentially be used to monitor therapeutic hyperthermia (41° C.).

Although preferred embodiments of the invention have been described herein, it will be understood by those skilled in the art that variations may be made thereto without departing from the spirit of the invention or the scope of the appended claims. All documents disclosed herein are incorporated by reference.

### REFERENCE LIST

Bell, A. G. (1880). On the production and reproduction of sound by light. Am J Sci 20, 305-324.
Chitnis, P. V., Mamou, J., McLaughlan, J., Murray, T., and Roy, R. A. (2009). Photoacoustic thermometry for therapeutic hyperthermia. Paper presented at: Ultrasonics Symposium (IUS), 2009 IEEE International.
Esenaliev, R. O., Oraevsky, A. A., Larin, K. V., Larina, I. V., and Motamedi, M. (1999). Real-time optoacoustic monitoring of temperature in tissues. 268-275.
Fofang, N. T., Grady, N. K., Fan, Z., Govorov, A. O., and Halas, N.J. (2011). Plexciton Dynamics: Exciton-Plasmon Coupling in a J-Aggregate-Au Nanoshell Complex Provides a Mechanism for Nonlinearity. Nano Letters 11, 1556-1560.
Kawabe, Y., and Kato, S. (2011). Influence of DNA on J-aggregate formation of cyanine dyes. 81030D-81030D.
Kim, G., Huang, S.-W., Day, K. C., O'Donnell, M., Agayan, R. R., Day, M. A., Kopelman, R., and Ashkenazi, S. (2007). Indocyanine-green-embedded PEBBLEs as a contrast agent for photoacoustic imaging. Journal of Biomedical Optics 12, 044020-044020.
Knoester, J. (2003). Frenkel and Charge Transfer Excitons in Electronic Solids. In Electronic Excitations in Organic Based Nanostructures, G. F. B. Vladimir M. Agranovi\ddot{c}, ed. (San Diego, Elsevier), pp. 487.
Kruger, R. A. (1994). Photoacoustic ultrasound. Medical Physics 21, 127-131.
Kruger, R. A., Liu, P., Fang, Y. R., and Appledorn, C. R. (1995). Photoacoustic ultrasound (PAUS)—Reconstruction tomography. Medical Physics 22, 1605-1609.
Lovell, J. F., Jin, C. S., Huynh, E., Jin, H., Kim, C., Rubinstein, J. L., Chan, W. C. W., Cao, W., Wang, L. V., and Zheng, G. (2011). Porphysome nanovesicles generated by porphyrin bilayers for use as multimodal biophotonic contrast agents. Nat Mater 10, 324-332.
Mo, G. C. H., and Yip, C. M. (2009). Supported Lipid Bilayer Templated J-Aggregate Growth: Role of Stabilizing Cation-π Interactions and Headgroup Packing. Langmuir 25, 10719-10729.
Oraevsky, A. A., Jacques, S. L., and Tittel, F. K. (1997). Measurement of tissue optical properties by time-resolved detection of laser-induced transient stress. Appl Opt 36, 402-415.
Pramanik, M., and Wang, L. V. (2009). Thermoacoustic and photoacoustic sensing of temperature. Journal of Biomedical Optics 14, 054024-054024.
Prokhorenko, V. I., Steensgaard, D. B., and Holzwarth, A. R. (2003). Exciton Theory for Supramolecular Chlorosomal Aggregates: 1. Aggregate Size Dependence of the Linear Spectra. Biophysical journal 85, 3173-3186.
Scheblykin, I.g. (2012). Temperature Dependence of Exciton Transport in J-aggregates. In J-aggregates, T. Kobayashi, ed. (Singapore, World Scientific Publishing Co. Pte. Ltd.), p. 325.
Shah, J., Park, S., Aglyamov, S., Larson, T., Ma, L., Sokolov, K., Johnston, K., Milner, T., and Emelianov, S. Y. (2008). Photoacoustic imaging and temperature measurement for photothermal cancer therapy. Journal of Biomedical Optics 13, 034024-034024.
Walker, B. J., Nair, G. P., Marshall, L. F., Bulović, V., and Bawendi, M. G. (2009). Narrow-Band Absorption-Enhanced Quantum Dot/J-Aggregate Conjugates. Journal of the American Chemical Society 131, 9624-9625.
Wang, L. V. (2009). Multiscale photoacoustic microscopy and computed tomography. Nat Photon 3, 503-509.
Wang, L. V., and Hu, S. (2012). Photoacoustic Tomography: In Vivo Imaging from Organelles to Organs. Science 335, 1458-1462.
Wang, X., Pang, Y., Ku, G., Xie, X., Stoica, G., and Wang, L. V. (2003). Noninvasive laser-induced photoacoustic tomography for structural and functional in vivo imaging of the brain. Nat Biotech 21, 803-806.
Zakharova, G. V., and Chibisov, A. K. (2009). Photostability of pseudoisocyanine J aggregates in polymer films and ways of its improvement. High Energy Chem 43, 294-297.

1. A nanovesicle having a bilayer comprising (i) a saturated first phospholipid and (ii) no more than about 15 molar % of a second phospholipid covalently conjugated to a J-aggregate forming dye.

2. The nanovesicle of claim 1, wherein the dye is selected from the group consisting of pseudoisocyanine, merocyanine, bis(2,4,6-trihydroxyphenyl)squaraine, Zn-chlorin, tetrtakis(4-sulfonatophenyl)-porphyrin, bacteriochlorin, antimony(III)-phthalocyanine, copper phthalocyanine and perylene bismide, Hypericin, subphtalocyanine, preferably bacteriochlorin.

3. The nanovesicle of claim 2, wherein the second phospholipid covalently conjugated to the J-aggregate forming dye is bacteriochlorophyll-lipid.

4. A nanovesicle having a bilayer comprising (i) a saturated first phospholipid and (ii) a second phospholipid covalently conjugated to a J-aggregate forming dye, wherein the dye does not comprise a porphyrin moiety.

5. The nanovesicle of claim 4, wherein the dye is selected from the group consisting of pseudoisocyanine, merocyanine, bis(2,4,6-trihydroxyphenyl)squaraine, Zn-chlorin, antimony(III)-phthalocyanine, copper phthalocyanine and perylene bismide, preferably bacteriochlorophyll.

6. The nanovesicle of claim 1, wherein the second phospholipid is present in the bilayer in an amount of between 0.01-15 molar %.

7. The nanovesicle of claim 6, wherein the second phospholipid is present in the bilayer in an amount of between 2-13 molar %.

8. The nanovesicle of claim 1, wherein the second phospholipid is present in the bilayer in an amount of about 5 molar %.

9. The nanovesicle of claim 1, wherein the second phospholipid is present in the bilayer in an amount of about 10 molar %.

10. The nanovesicle of claim 1, wherein the second phospholipid is present in the bilayer in an amount of about 15 molar %.

11. The nanovesicle of claim 1, wherein the second phospholipid is selected from the group consisting of phosphatidylcholine, phosphatidylethanoloamine, phosphatidylserine, phosphatidylinositol, lyso-phosphatidylcholine, lyso-phosphatidylethanoloamine, lyso-phosphatidylserine and lyso-phosphatidylinositol.

12. The nanovesicle of claim 11, wherein the second phospholipid comprises an acyl side chain of 12 to 22 carbons.

13. The nanovesicle of claim 1, wherein the dye is conjugated to the glycerol group on the second phospholipid by a carbon chain linker of 0 to 20 carbons.

14. The nanovesicle of claim 1, wherein the saturated first phospholipid is selected from the group consisting of phosphatidylcholines, phosphatidylethanolamines, phosphatidic acid, phosphatidylglycerols and combinations thereof.

15. The nanovesicle of claim 14, wherein the saturated first phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG), 1,2-dipentadecanoyl-sn-glycero-3-phosphocholine (PC(15:0/15:0)), 1,2-diheptadecanoyl-sn-glycero-3-phosphocholine (PC(17:0/17:0)), 1,2-dinonadecanoyl-sn-glycero-3-phosphocholine (PC(19:0/19:0)), 1,2-diarachidoyl-sn-glycero-3-phosphocholine (PC(20:0/20:0)), and combinations thereof.

16. The nanovesicle of claim 1, further comprising PEG-lipid.

17. The nanovesicle of claim 16, further comprising DPPE-PEG2000.

18. The nanovesicle of claim 16, further comprising DSPE-PEG2000.

19. The nanovesicle of claim 16, wherein the PEG or PEG-lipid is present in an amount of about 5 molar %.

20. The nanovesicle of claim 1, wherein the nanovesicle is substantially spherical and about 110 nm in diameter.

21. A method of monitoring temperature at a target site comprising:
a. providing the nanovesicle of claim 1 at the target site,
b. monitoring absorbance at the target site,
wherein a blue shift in absorbance is indicative of temperature at the target site being higher than a predetermined temperature, the predetermined temperature corresponding to a transition temperature of the saturated first phospholipid, and
wherein a red shift in absorbance is indicative of temperature at the target site being lower than the predetermined temperature.

22. A method of monitoring temperature at a target site comprising:
a. providing the nanovesicle of claim 1 at the target site,
b. monitoring a photoacoustic signal at the target site,
wherein a lack of a photoacoustic signal is indicative of temperature at the target site being higher than a predetermined temperature, the predetermined temperature corresponding to a transition temperature of the saturated first phospholipid, and
wherein a presence of a photoacoustic signal is indicative of temperature at the target site being lower than the predetermined temperature.

23. A method of monitoring temperature at a target site comprising:
a. providing the nanovesicle of claim 1 at the target site,
b. monitoring a fluorescence signal at the target site,
wherein a presence of a blue shifted fluorescence signal is indicative of temperature at the target site being higher than a predetermined temperature, the predetermined temperature corresponding to a transition temperature of the saturated first phospholipid, and
wherein a presence of a red-shifted fluorescence signal is indicative of temperature at the target site being lower than the predetermined temperature.
